(19) United States
(12) Patent Application Publication
(10) Pub. No.: US 2012/0282682 A1
(43) Pub. Date: Nov. 8, 2012

(54) MICRO-CHANNEL STRUCTURE METHOD AND APPARATUS

(75) Inventors: Peter Walsh, Hemel Hempstead (GB); David Albin, Hemel Hempstead (GB); Martin Gouch, Hemel Hempstead (GB)

(73) Assignee: FFEI LIMITED, Hemel Hempstead, Hertfordshire (GB)

(21) Appl. No.: 13/505,417

(22) PCT Filed: Oct. 28, 2010
(86) PCT No.: PCT/GB2010/051808

§ 371 Date: Jul. 23, 2012

(30) Foreign Application Priority Data
Nov. 2, 2009 (GB) 0919207.1
Nov. 4, 2009 (GB) 0919377.2

(51) Int. Cl. B81C 1/00; B32B 37/00; B05D 1/02; B05D 3/00; C12M 1/34; B01L 3/00

(52) U.S. Cl. 435/287.2; 422/503; 427/421.1; 427/372.2; 156/60; 29/458

(57) ABSTRACT

A method is provided of forming a micro-channel structure for use in a biosensing device. A master structure is provided having a first configuration of micro-channels with respective first fluid flow characteristics. One or more regions of material are deposited onto the master structure using a fluidjet process so as to modify the first configuration into a second configuration having respective second fluid flow characteristics, different from the first. Functional biosensing devices formed using the method are also described.

### FIELD OF THE INVENTION

The present invention relates to a method for forming a micro-channel structure for use in a biosensing device.

### BACKGROUND TO THE INVENTION

Biosensing devices such as immunoassays which utilise micro-channels fabricated into or onto a carrier are finding increasing utilisation in the field of biotechnology. A discussion of microfluidic platforms is provided by S Haeberle and R Zengerle in RSC LabChip 2007, 7 pp 1094-1110. Such assay or Lab-on-Chip devices enjoy relatively widespread use, or at least uses for such devices have been often proposed. The method of fabrication may be by moulding (see for example U.S. Pat. No. 6,039,897), by the application of a precursor that is then masked and heat treated, or by number of other methods.

Numerous methods are already known for detecting molecules (reagents), their immobilisation and the effect of introducing analytes thereto. The potential for the application of biosensing devices is growing at a significant rate now that the necessary technologies exist in, at least, the laboratory. The range of applications means that, especially, the science relating to the analyte/reagent interaction will be ongoing for some considerable time and it will be of great assistance in this development to have the ability to rapidly prototype and pilot the effectiveness of biologic combinations for the detection of target analytes in a medium and thereafter rapidly put into manufacture sensors that can be used in the real world.

The application drivers for biosensing devices require both qualitative and quantitative detection where qualitative detection is often stated as a sufficient requirement because the cost of a quantitative test is perceived as too high.

In the light of these advances there is a need to provide simple and commercially practical manufacturing methods and techniques which allow the production of known and new biosensors at a low cost.

### SUMMARY OF THE INVENTION

In accordance with a first aspect of the present invention, we provide a method of forming a micro-channel structure for use in a biosensing device, comprising:—

a) providing a master structure having a first configuration of micro-channels with respective first fluid flow characteristics; and,
b) depositing one or more regions of material onto the master structure using a fluidjet process so as to modify the first configuration into a second configuration having respective second fluid flow characteristics, different from the first.

The present invention allows the design of inexpensive, disposable, personal and sensitive devices for detecting and quantifying analytes in a medium, where the medium, bioactive reagent, immobilised molecule or probe, together with processing of the target analyte, are all customizable at the point of manufacture. The present invention provides for manufacturing methods and processes suitable for producing both small and large quantities, outside of the laboratory, of biosensing devices.

The invention achieves this by the provision of a master structure of micro-channels which provides a common building block to numerous biosensing devices, a biosensor of each such device being provided with a particular specificity. The micro-channels are therefore “programmable” in the sense that the fluid flow characteristics of the micro-channels are controllable by the deposition of fluidjet material in one or more locations within the master structure. The fluid flow characteristics may control the actual flow path or paths taken by the fluid within the structure. Thus fluid applied to the master structure under the first configuration may flow along a different set of paths to those taken under the second configuration. The fluid flow characteristics may also dictate the conditions under which such fluid will begin or end flowing, or the manner in which the fluid flows within the structure (such as its flow rate).

More generally the method relates to the fabrication of micro-channel structures for biosensing devices which may be customised by defining the characteristics required for a specific biosensing device; such characteristics including introduction of the medium containing the analyte, necessary illumination paths, flow paths, surface treatments, flow-determining artefacts and chambers where immobilised reagents are located.

The micro-channels include a number of different types of structure including conduits, valves, and chambers. The micro-channels define small flow paths that may include reagent location sites, constrictions, valves, pumps, and mixing chambers. Such structures may utilise capillarity as the mechanism for moving the applied fluid medium with the movement being by detailed channel design and control of the contact angle. Alternatively or in addition a flow wave could be induced by pumping, depression of a bladder/bellows or vibration (including shaking). The driving force could also be achieved by an osmotic process that utilises a semi-permeable membrane between medium introduction and reagent location.

The design and production of the micro-channels includes control of the aspect ratios and cross sectional profiles of the component features which are typically in the size range of 5 μm to 300 μm. In most applications the length of individual micro-channels may be up to 50 mm. Optionally, the micro-channels may be layered and interconnected in the Z axis by such methods as vias. In this case the micro-channel structures may be layered together following after the programming step Additionally it is possible to fabricate variable paths by changing the width/radii ratio of the micro-channels. An example is shown in FIG. 13 (given that the capillary transport distance is a function of 1/r). By such means a continuously tapering channel from analyte to reagent would require less precise control of the contact angle.

The micro-channels may be fabricated in master structures formed from impermeable (non-wicking) substrates although they may also be formed in master structures having fibre structures or other ‘wicking’ materials. Separately placed fibre structures may also be provided. Fibrous materials may therefore provide part or all of the capillary flow paths and other fluidic artefacts that are required. This may be by barrier structures of specifically tailored wetting fibres (such as hemi cellulose) or constructed fibre bundles for example. This includes the option to combine these features with those of non-fibre substrates.

The substrate is the physical material upon or within which the elements of the biosensing device are contained and within which the master structure is preferably formed. The substrate is capable of being both monolithic in nature or a complex construction of different elements. A number of different substrates are contemplated including: Naturally fibrous paper or board; Constructed fibrous material that contains a designed and oriented arrangement of one or more of cellulose, hemi cellulose and lignin; the result of a polymerisation process (either in situ or previously formed) such as MMA (methyl methacrylate), PMMA (poly methyl methacrylate), PDMS (poly dimethyl siloxane), PLA (poly lactic acid), or lactide/glycolide copolymers, Polyimide, Other homo- or heteropolymers that could be used to provide specific physical properties, such as flexibility, chemical resistance, refractive index, and so on; and, Glass frit (granulated glass). A key property of the substrate is that, if in contact with the analyte or reagent, it must not cause the denaturing or otherwise harm either the analyte or reagent. This can be achieved by substrate arrangement or chemically, or by a combination of each of these.

The modification of the first configuration of micro-channels so as to form the second configuration is performed using a fluidjet process. Such a process is typically a non-contact process in which the material is passed from a jetting head to a target location upon the micro-channel arrangement. Typically inkjet technology can be used for this purpose. The principal requirements of the jetted material is that it bonds sufficiently to the master structure and is chemically and physically robust so as to form, for example, impenetrable barriers for flow diversion under the range of temperature, pressure, and humidity conditions under which the fully formed biosensing device might be used. The specific chemical resistance of the deposited material will necessarily vary depending on the analytes used within the biosensor, but it is imagined that the permanent nature of the modification process would preclude use of water soluble or particularly hygroscopic materials. Rather, it is preferred that the materials used in the configuration process are chosen from polymeric or polymerisable components. The former classification would include thermoplastic polymers whose glass transition temperature (Tg) was above that of the maximum practical temperature at which the biosensing device would be used, and below the maximum operational temperature of the fluidjet process. For inkjet piezo deposition this range would therefore be between 60° C. and 140° C., although those versed in the art will realise that other fluidjet deposition systems are capable of extending this upper boundary temperature.

In terms of a polymerisable component, it can be assumed that this could conceivably include simple two-part epoxy curing systems, as well as those processes which require an additional external source to provide initiation. This would include UV cured free-radical or cationic systems, thermally initiated crosslinking systems (for example using peroxides), electron beam cured fluids, and so on. As a particular example a UV cured acrylate monomer solution containing an activating photoinitiator could be deposited by inkjet onto a preformed fibre substrate. The viscosity of the applied solution and the wetting parameters of the liquid and the substrate will determine the degree of penetration of the liquid into the fibre material and its flow across the surface prior to the curing step.

The fluidjet process is one in which controlled but variable amounts of the fluid or fluids used in the construction are deposited in the required spatial arrangement by non-contact methods. Generally the jetting methods will utilise currently available industrial components with deposition systems that are either binary or greyscale in method and of variable resolution; terms well understood to those versed in the art of fluidjet technology. However the associated techniques of microjetting and continuous inkjet are also included as application processes as they provide specific methods for placing larger “dots” of material and can also cater for different fluid properties.

Herein the “fluidjet material” describes fluid materials that are used for construction of the biosensing device and is not intended to refer to reagents, the medium or analytes, although these may also be fluidic in nature. The chemical nature of the construction fluids will necessarily vary depending on the task envisaged and the application technique involved in this step. For example, a material for piezo drop-on-demand jetting will have specific physical properties (such as viscosity, surface tension, particle size) to enable it to perform reliably in the system. Typical carriers employed in the art (such as solvents, oils, water, or UV monomers) could be adapted to modify the construction fluids for a particular application purpose. Alternatively, the use of a ‘phase change’ fluid, such as paraffin waxes, low melting thermoplastics is contemplated. Phase change materials in this context are assumed to be liquids of a jettable viscosity under conditions of elevated temperature. Once these fluids are deposited onto a substrate, they are imagined to solidify and remain so under the environmental conditions of use for the biosensing device. Practically for a typical piezo inkjet system this would imply a jetting temperature between 60° C. and 140° C., although those versed in the art will understand that other fluidjet deposition devices exist to extend the upper operating range.

Once applied, the accumulated jetted fluids can be converted to a more permanent solid species by a curing process. The jetted fluid material may be arranged to “self-cure” by a natural chemical process inherent in the fluid itself (such as oxidation) or a natural chemical process resultant from a defined mixing of fluids after deposition (for example polymerisation). However, as an alternative an additional curing treatment may be applied to the deposited material, such curing occurring by Irradiation by controlled spectra (such as UV, IR) or an electron beam for example.

The fluid jet material may be provided to the master structure so as to block a particular micro-channel or channels. Alternatively, it may be arranged to partially block or restrict the one or more micro-channels which may have an effect such as reducing the local flow rate of fluid past the restriction or act as a valve which only allows passage of the fluid once there is a sufficient driving force (such as pressure) to do so.

A number of different master structures are contemplated. It is desirable, for example, in many applications to provide a number of similar biosensing functions upon the same “chip”. This may be achieved by providing the micro-channels arranged into a plurality of sets. Each set may comprise one or more micro-channels such that the arrangement of the micro-channels is the same within each set (thus providing multiple instances of the same sensor for example). The sets of micro-channels may be arranged side-by-side in an array. Such an array may be two-dimensional (or three dimensional if stacking of the structures is effected). The sets of micro-channels are preferably arranged to be separate from one another in that no fluid path exists between them. The sets allow the provision of an array of biosensing devices having an identical function and therefore allowing multiple samples to be tested simultaneously or serially upon the same chip. One advantage of the master structure is that it allows individual sets of micro-channels to be provided with different biosensing functions, namely a different specificity. Thus a number of separate tests may be performed upon the same chip which may be related tests (such as by controlled variables including reagent quantities, analysis times, reagent types) or indeed the tests may be entirely unrelated. Thus one or each of the first configuration (the original unmodified master structure) or second configuration (the master structure as modified by the fluidjet material) may provide a plurality of fluid flow paths which are physically isolated from each other so as to allow a different biosensing function to be performed by each fluid flow path. Each individual set of micro-channels may be formed as a biosensor with the different biosensors forming a biosensing device (this term including the provision of only a single biosensor).

When in use the “biosensing device” functions by providing an analyte to a reagent, the interaction of these entities causing a measurable response if the analyte has a certain composition or properties. Although the discussion herein is generally in relation to an analyte medium begin provided to an immobilised reagent, in principle the reagent may be mobile and the analyte immobilised.

The analyte is a substance or constituent that is required to be determined in an analytical procedure and can comprise any of antibodies, antigens, biomarkers or any other cell, biological molecule or combination thereof that is capable of specificity and is of interest to detect. The analyte is carried within a medium.

The medium is typically a liquid carrier containing the analyte. The medium may be naturally liquid at NTP (normal temperature and pressure) or which contains material which has been mechanically scavenged or macerated and then suspended to achieve the necessary fluidic properties at NTP. This carrier could also be considered as being contained within the biosensor system and acting as an eluent. In this case, this fluid is considered to be miscible with the analyte or a specific portion thereof.

The reagent is typically a constructed antibody, molecule or probe or other biological molecule that may have indicator chemistry or molecules conjugated thereto; for example a fluorescent or colorimetric indicator. The reagent is designed to have specificity to one of the contents of the analyte of interest. The reagent is generally presented by a support which is a physical feature or chemical treatment that serves to isolate and present the various reagents so that they can intimately contact and react with the analyte. The reagent is typically immobilised in that the reagent is placed in specific locations within the micro-channels and will not migrate away from its placed location. Immobilisation techniques include the controlling of the surface activity of the location site.

Typically the interaction between the analyte and the reagent results in an optical artefact. The actual nature of the optical artefact will be dependent upon whether the reagent has been conjugated with an indicator and what the specific indicator is (for example fluorophore). Many indicators require that the analyte/reagent mix is illuminated by spectral radiation thus causing a subsequent emission of spectral energy giving a colorimetric response and effect that is within the spectral region from 350 nm to 800 nm. This may be simple fluorescence, fluorescence resonant energy transfer (FRET) or by bioluminescence or, indeed, simple colorimetric change without excitation. The common requirement for the optical artefact is that the yield will be proportional to the amount of active species in the analyte, i.e., a quantitative response and there will be measurable energy and colorimetric (spectral) data resulting from the process.

Following the provision of the modified master structure or after the provision of the second configuration, the method preferably further comprises applying a surface treatment to one or more regions of the micro-channels so as to affect the wettability characteristics of the said one or more regions. The surface treatment may be used for the modification of the surfaces, both during construction and thereafter, to assist and control the movement of the medium through the micro-channels. This is also often described as controlling wetting behaviour. During manufacture this term is intended to convey methods such as modification of the surface energy at different stages of manufacture by corona discharge, air or gas plasma treatment, laser patterning, irradiation by a controlled spectrum (such as UV) or chemical treatment. For the purposes of use of the biosensing device (rather than manufacture), the surface treatment controls the hydrophilic/hydrophobic or oleophilic/oleophobic nature of the flow surfaces and support/immobilisation points using similar techniques to those listed as for manufacturing.

In addition to the production of the master structure having the second configuration (as provided by the fluidjet process), the method further comprises providing reagent to at least one of the microchannels for use in a biosensing function. Typically the reagent is immobilised by this process or by an additional subsequent process. Preferably the reagent is provided by a fluidjet process. The reagent may be applied using the same fluidjet apparatus as is used to provide the second configuration in step (b). Thus the same jetting head may be used to provide the combined function, albeit with independent jet nozzles.

The micro-channels are enclosed by the application of a further sealing layer which can be practically imagined to be a lamination step using heat and pressure to apply a sheet of, for example, a thermoplastic polymer (such as polymethyl methacrylate) over the entire surface. Other covering processes such as forming a top surface using similar embossing steps previously mentioned and then gluing or laminating could also be imagined. Additionally, a fibre substrate could be structured and arranged so that it could be folded and sealed at a later stage.

In order to assist in the detection of an optical artefact the method may further comprise the provision of an optical element to the structure. Such an optical element may be one or more of a lens, waveguide, light pipe or grating. Illumination is used to make visible, stimulate or excite the conjoined analyte and reagent(s). The spectral content (for example being between 320 nm and 700 nm) of the illumination source and its intensity can be variable by means of selection of different lasers, LED, incandescent or other sources and also in combination with filters, such methods being well understood by those versed in the art. The source of illumination could also be conceived as being internally created, for example, in the form of bioluminescence or quantum dots. The inclusion in the overall biosensing device structure, of one or more of waveguides, light pipes, lenses and gratings allows the illumination (source in the detection instrument) to be delivered to the reaction points.

Detection of the analyte-reagent interaction is made by the observation and measurement of the resultant optical artefact created by the reagent coming into contact with the analyte. An important aspect of detection is that the resultant optical artefact may be of low energy and further may be masked by background “noise” effects. However, methods of cascading adjacent multiple test cells or ‘standard addition’ procedures could also be used to reduce background effects. If and when necessary, a small digitally defined lens may be applied by jetting a fluid over the observation point or points in the reaction chamber (or chambers) to enhance the optical artefact; and indeed to define to a user, not skilled in the use of the device, the location of interest. The fluidjet deposition of such lenses will convey all the benefits of this production process, such as, accurate placement, small and scalable sizes. This process has been previously demonstrated for a Microjet dispensing system. For qualitative results, visible (to the eye) colour, or a gradation thereof may be sufficient but it is foreseen that a significant benefit of such biosensing devices is in the quantitative determination of analyte species where specific measurement is required. The present invention intends that the capture of the optical artefact can be by means of a simple instrument (the limiting case of which is the camera in a cell phone).

Thus, the biosensing device is the complete device that has at least a means to receive the medium, conjoin it with one or more reagents and make the results visible to the eye or an instrument as applicable to the specific embodiment. Such a device can contain one or many test mechanisms for the same or different analytes and which may be used together, serially or not at all. The present invention, consequent on the design and manufacturing methods, includes a complete biosensing device.

A second aspect of the invention therefore includes a micro-channel structure formed using the method of the first aspect. A third aspect of the invention comprises a configured micro-channel structure for use in a biosensor, comprising a master structure having a first configuration of micro-channels and one or more regions of fluidjet deposited material applied to the micro-channels so as to provide a second configuration in which the fluid flow characteristics of the master structure are modified. Preferably the micro-channel structure is provided with an immobilised reagent, and a supply device for providing an analyte to the reagent.

The biosensing device may be provided with a further artefact such as a removable cap that can be rearranged to cause a permanent one-time compression of an liquid or gas storage mechanism which can then expel a controlled volume and at a controlled rate of liquid/gas with the resulting liquid/gas flow into the micro-channel matrix initiating the analyte coming into the necessary proximity with the reagent. As an alternative or in addition, the cap may be rearranged to serve as a stand for the biosensing device thus determining the spatial orientation; then allowing gravity to initiate the analyte coming into the necessary proximity with the reagent.

A bellows or bladder may also be utilised with the micro-channel arrangement. These may effectively sit on top of the device which is then compressed by the removable cap. In operation a protector may be provided to prevent the bellows from being compressed accidentally. The bellows may alternatively be placed in a recessed area with a upper surface of the bellows flush with the generally planar upper surface of device. Then if the removable cap has a fixed volume of protrusion when it was pressed over the bellows it will deliver a fixed amount of gas or liquid. The fixed volume within the protrusion could be fluidjetted into the removable cap in order to provide a configurable function. The bellows may also be “oversized”, that is, protruding over the edge of the device. It may then be compressed once with the removable cap or another flat surface or the removable cap may be formed so that it fits into the bellows recess. The calibration may be performed upon the removable cap or identically shaped stamp and then jetted onto the removable cap. As a further alternative the removable cap may be recessed and the bellows project on top of the device “slide”.

In general, a delivery system may be used within the device, the type of such a system depending upon the particular application in question. The delivery system provides an applicable method to improve the transport or transfer of the analyte/medium through the various channels, mixing chambers, and so on, of the biosensing device. This may include application of manual methods such as a ‘one use’ air pocket to deliver a specific volume pulse, or a bellows-type bladder as discussed above. Alternatively powered micropumps (fabricated from, for example, PMMA/PDMS) can be fabricated into the flow channels at specific points for fluid delivery. In all of the preceding methods, the design and control of wetting behaviour and the use of surface modification both globally, zonally or at specific locations (such as hydrophobic plugs) is a key element of the system.

An optional activation method may be used to initiate delivery of the medium to the reagent. The biosensing device may optionally comprise a slip cover, which is separate or conjoined and articulated, into which the sensor is engaged after introduction of the medium, such engagement cause pressure to be applied to a bladder or pump and thus initiate flow. An alternative method is a different mechanism, either separate or built in and articulated whereby, before or after introduction of the analyte, the spatial orientation of the sensor is determined and fixed; for example to the vertical, thus using gravity to initiate flow.

### BRIEF DESCRIPTION OF THE DRAWINGS

Some examples of a method of forming a micro-channel structure and a corresponding device are now described with reference to the accompanying drawings, in which:—

FIG. 1 shows a first example arrangement of micro-channels;

FIG. 2 shows a second example arrangement;

FIG. 3 shows how deposited material may affect the configuration of the channels of the first example arrangement;

FIG. 4 shows a third example arrangement of micro-channels;

FIGS. 5a to e illustrate schematic side views, partly in section, of micro-channels including restrictions;

FIGS. 6a to c show example end elevations of the channels;

FIG. 7 illustrates the provision of a region of modified surface energy affecting hydrophobicity;

FIG. 8a shows a first example bio-sensing device;

FIG. 8b shows the first example device viewed from one end;

FIG. 9 shows the device with an end cap removed;

FIG. 10 shows the use of the end cap to activate the device;

FIG. 11 shows the use of the end cap to provide gravitational activation;

FIGS. 12a to d show how bellows may be calibrated and used;

FIG. 13 is a schematic illustration of a tapered micro-channel;

FIG. 14 is a flow diagram of an example method for producing an ELISA biosensing device;

FIG. 15 illustrates the used of jetted material to form a mixing chamber; and,

FIG. 16 shows how deposited material may be used to control the reaction period.

### DESCRIPTION OF PREFERRED EXAMPLES

The present invention, describes a complete biosensing device fabricated on and/or within a substrate, said biosensor comprising a mechanism to introduce the medium, a micro-channel structure, support, and immobilisation for the included reagent or reagents and a delivery system that, optionally in conjunction with the activation artefact will deliver controlled amounts of the medium to one or more reagents in the following combinations and such that they can be then detected:

i) a single amount of the medium is delivered to a defined concentration of a single reagent;
ii) different amounts of the same medium are delivered to a defined concentration of multiple reagents;
iii) multiple and equal amounts of the same medium are delivered to defined but different concentrations of the same reagent;
iv) and all possible combinations thereof.

The biosensor additionally contains the necessary illumination structure and other artefacts that are required for initiating flow such as, but not limited to, bladders and micro-pumps or the activation system. The biosensor can then subsequently be read on an instrument designed for the purpose.

A method for forming a biosensor is now described, beginning with a discussion of the master structure and programming concepts.

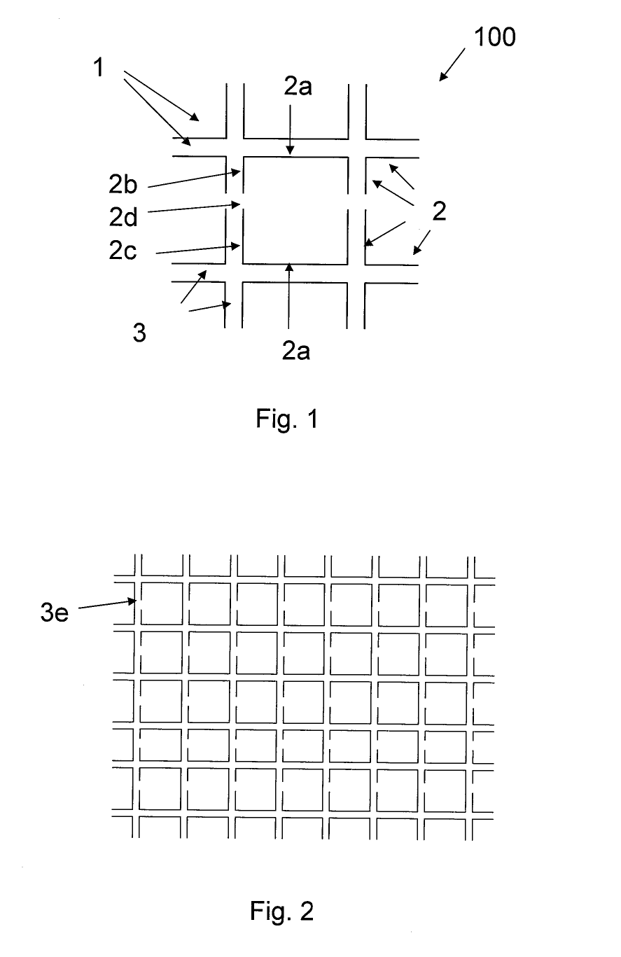

A first example of a master structure 100 is indicated in FIG. 1. This comprises a polymer substrate 1 upon which are formed a number of ridges 2. The formation of the master structure may be effected by a number of known techniques depending upon the material in question. In the present case the structure is embossed. The ridges 2 are arranged in a pattern which repeats in two dimensions across the surface of the master structure 100. Thus a unit cell of the structure may be described as a square. Each repeating unit cell comprises a generally square arrangement of ridges which project away from the generally planar surface of the substrate 1. Each cell comprises two opposing unbroken walls of substrate material 2a forming first opposed sides of the square. The other two sides are provided by two broken walls of material (each wall comprising parts 2b and 2c with a gap 2d therebetween). The elongate regions between the ridges 2 in adjacent cells defines channels 3. In FIG. 1 a typically width of the channels is 100 micrometres, with the length of the square sides 2a being about 1000 micrometres. If a suitable fluid is introduced into the channels 3 then, provided the surface energy of the liquid-substrate interface is an appropriate magnitude then the fluid is able to flow within the structure filling the channels and also passing through the gaps 2d to fill the interior of the squares bounded by the ridges 2a,2b,2c. Thus the entire structure may be flooded with such a fluid under these circumstances.

Referring now to FIG. 2 an alternative master structure is presented. In comparison with FIG. 1, FIG. 2 shows a larger number of unit cells arranged in a two dimensional array. The size of the unit cell in FIG. 2 may be the same of different from that of FIG. 1. The key distinction between the unit cell square of FIG. 1 and FIG. 2 is that in FIG. 2 only one of the ridge walls 2 within each square is provided with a gap, which in this case is referenced as 3e. Thus the interior of each square of walls is accessible to a fluid via a single entry point at 3e. This results in a “blind” or closed path within which the fluid may accumulate.

Although the ridges 2 in FIGS. 1 and 2 are arranged in rectangular patterns it will be appreciated by those skilled in the art that this may not be the best arrangement for a desired fluid system. The proposed master arrangement concept is entirely flexible and might manifest itself in numerous other forms. One alternative example is shown in FIG. 4 in which again a square unit cell may be defined. In this case the ridges 2 are formed as a series of chambers 5 and interconnecting channels 6. The chambers 5 are arranged a square grid and each chamber has four conduits 5 leading to/from it, the positions of the four conduits being distributed evenly around the chamber walls. Rather than being arranged as rectilinear paths, each conduit 6 in this case is arranged in a serpentine fashion. One purpose of such a geometry is to increase the path length (and therefore the propagation time) of fluid passing along the conduits. Many other combinations, numbers of connecting conduits and shaped chambers are contemplated.

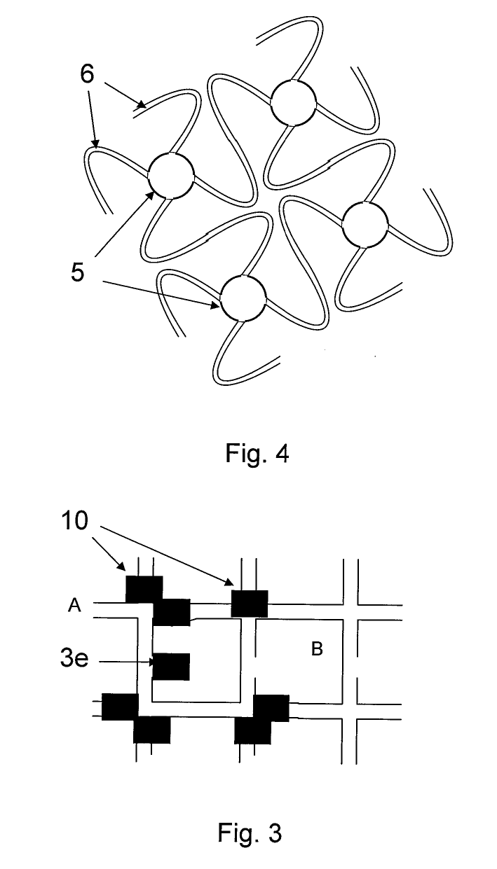

FIG. 3 illustrates schematically how the master arrangement of micro-channels may be programmed by the deposition of material using a fluidjet process. Thus a common master arrangement may be used in a number of biosensing devices. The modification of the fluid flow paths allows for the specific requirements for a particular sensor to be achieved by “programming” with the fluidjet material. Thus the master arrangement has an initial first configuration and this is then modified into a second configuration by the application of fluidjet material. Specified fluid flow paths may be established through the master structure by means of blocking channels as shown in FIG. 3.

In FIG. 3 the master structure configuration of FIG. 2 is modified by the deposition of a number of quantities of fluidjet material which are positioned at specified locations within the structure. In this case the fluidjet material completely blocks the local channel or gap within the master structure at the location within which it is placed. Thus the resultant configuration in FIG. 3 provides for a fluid (such as a medium bearing an analyte) to pass into the structure at point A. It is then diverted past a former gap 3e which is now blocked by the fluidjet material. The medium is then directed around two outer walls of the square whose entrance 3e was blocked and it is then diverted into the blind chamber at B where a reagent material may be immobilised for example. FIG. 3 demonstrates how the complete blocking of pathways in the micro-channels provides for the direction of the medium along a predetermined path.

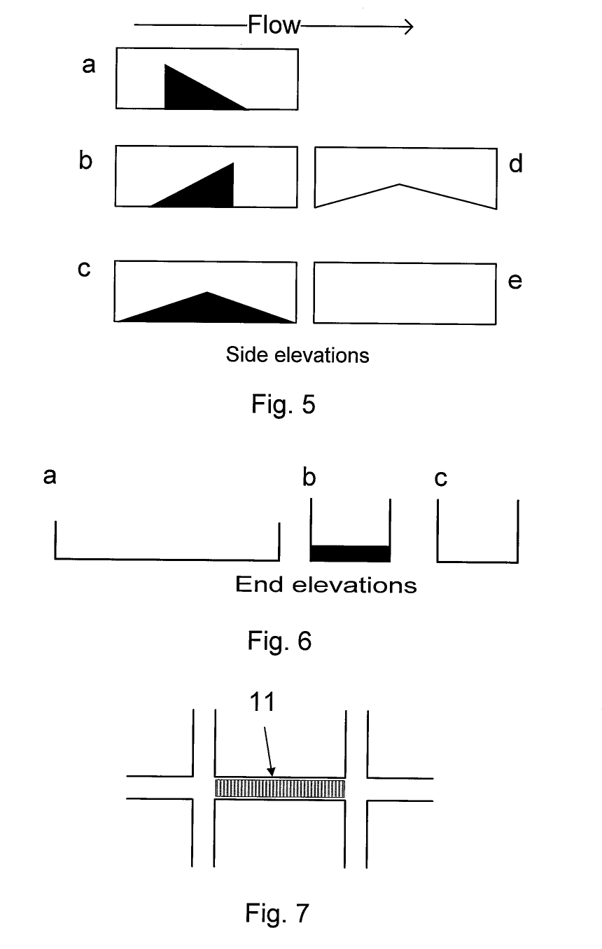

We refer now to FIGS. 5a to 5e which are schematic side views of restrictions within the micro-channels. In FIGS. 5a to 5c there is illustrated the manner in which partial blocking of a particular micro-channel may be effected by controlled deposition of fluidjet material. The direction of medium fluid flow is from left to right in each drawing. In FIG. 5a the fluid is presented with a stepwise reduction in the micro-channel geometry, followed by a ramped increase in dimension beyond the initial stepwise position. In FIG. 5b the opposite geometry is present with respect to the medium flow direction, namely a ramped narrowing of the micro-channel to a minimum dimension followed by a stepwise return to the full larger dimension. FIG. 5c illustrates “back to back” ramping in which the restriction ramps in magnitude to a minimum dimension followed by a ramping return to the full micro-channel dimension. It is also noted by way of example in FIG. 5d that the micro-channel itself may be formed with a localised restriction, the one illustrated in FIG. 5d being analogous to the geometry provided by the fluidjet material in FIG. 5c. FIG. 5e simply shows the full dimension of the micro-channel in an unrestricted part of the flow path. The restrictions illustrated in FIGS. 5a to 5d (and in FIGS. 6a to 6c below) allow control over the flow rate of the medium within the micro-channel.

FIG. 6 schematically illustrates end views of the micro-channels. In FIG. 6a a wide and shallow micro-channel is provided. In comparison, in FIG. 6b a significantly narrower and deeper channel is illustrated. In this case the channel is partially blocked with fluidjet material. FIG. 6c illustrates a micro-channel of similar dimensions in the absence of the fluidjet material.

Further control of the fluid flow characteristics of the medium may be achieved by changing the hydrophobic nature of some parts of a channel or chamber. This is illustrated in FIG. 7 which as an example restricts the flow of the medium until a specific differential pressure exists between the medium and a downstream area of the channel. This is achieved by applying a surface treatment to a localised region 11 of the micro-channels using a technique such as a laser treatment. It is moreover possible to use patterns of hydrophilic and hydrophobic treatments (or oleophilic/oleophobic treatments), for example within a chamber, to enable mixing of the analyte. This may be done alone or in conjunction with physical artefacts, which themselves might be surface treated, located within the same chamber.

Thus it is firstly possible to design rapid prototypes electronically, for example on a computer using software designed for the purpose by defining the specificity (such as routes, mixing and flow characteristics) of the micro-channel matrices; and thereafter to use the same data to program the master micro-channel structure and send to the production machinery for manufacturing the devices in any quantity. Preferably the entire process is under the control of a computer program operating high precision apparatus to perform each step of the method.

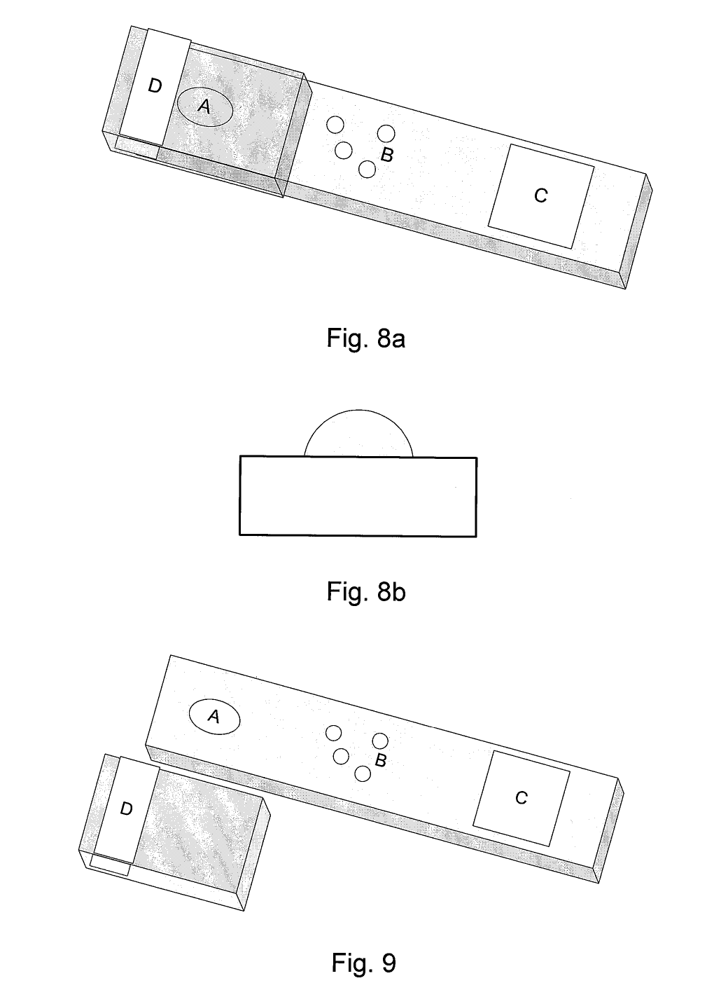

An example of a finished micro-sensing device produced using the method of the invention is shown in FIG. 8a. This takes a generally rectangular from. In FIG. 8a, a point for the introduction of a medium bearing an analyte is illustrated at A adjacent to one end of the device. A number of observation points are shown at B, each of which is provided with a small lens. The observation points are shown approximately halfway along the length of the device. In this case the device comprises a number of separate chambers each of which is provided with either a different reagent or a similar reagent which is either in a different local environment or which is reached by the medium from A under different local conditions. The lensed observation points are used by the detection system (not shown) to view the result of combining the analyte(s) and reagent(s) at the various locations. In this example multiple individual paths for the medium are provided between A and the locations B. An optional bellows device containing a defined amount of air or other gas is illustrated at C. The device is provided with a replaceable cap D which covers the end of the device. Removal of the cap exposes the entry point A for the medium. FIG. 8b simply illustrates an end view of the device with the blister/bellows design shown in its extended configuration. FIG. 9 shows the same device with the multipurpose removable cap D detached and the sensing device ready for introduction of the medium.

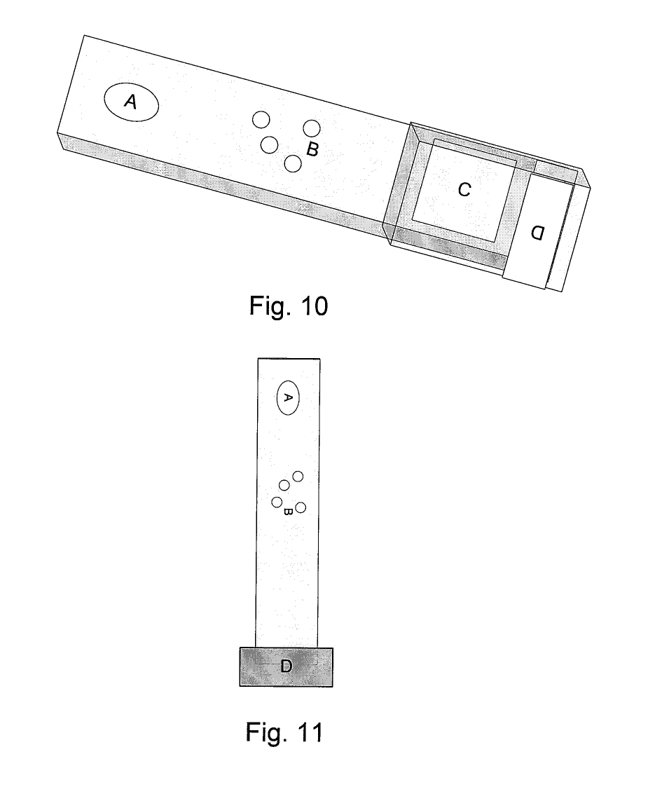

FIG. 10 shows same device with the multipurpose removable cap D fitted over the bellows end C. The depressing of the bellows C causes a permanent one-time compression of the bellows thus expelling a controlled volume and at a controlled rate of air/gas with the resulting air/gas flow into the micro-channel matrix initiating the medium's journey to the reagent. FIG. 11 shows an alternative embodiment whereby there are no bellows. The multipurpose removable cap D serves as a stand for the biosensing device determining the spatial orientation; then allowing gravity to initiate the medium's journey to the reagent. Those experienced in the art will see that these possibilities extend the capability of the devices when simple capillary forces are insufficient or are not the optimum method of flow but do not preclude the use of simple capillary flow only.

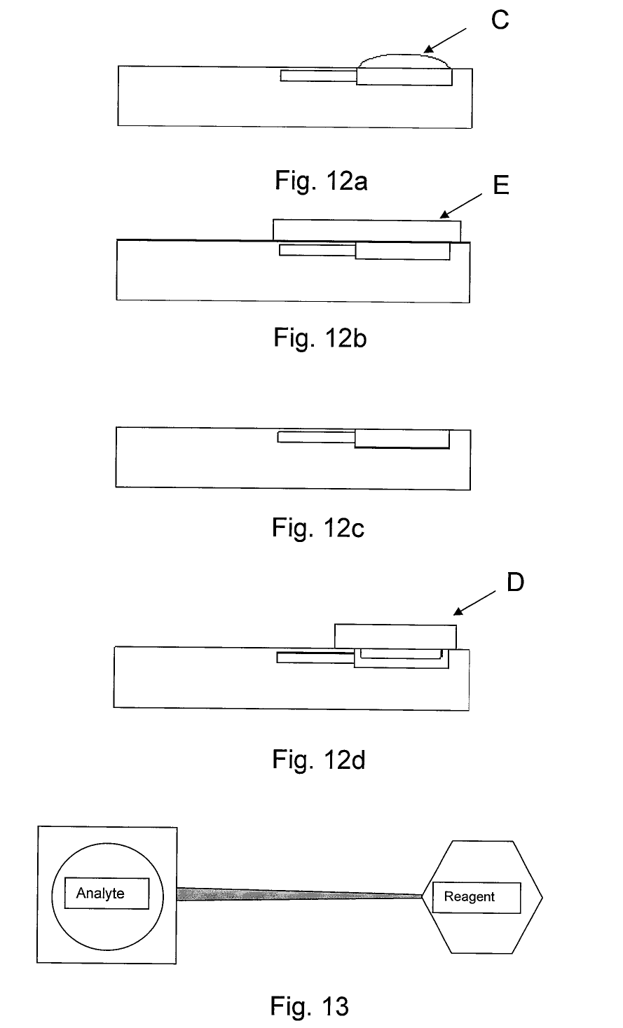

FIG. 12a shows a device having bellows C projecting slightly above the general plane of the upper surface of the device (the device being formed as a “slide”). A calibration of the bellows C is applied by placing a solid planar structure E over the end of the device so as to slightly compress the bellows C and ensure that it has a flat upper surface (see FIG. 12b). The device may be shipped to a customer in the calibrated state as shown in FIG. 12c. Later when in use, a predetermined volume reduction within the interior of the bellows (of equal volume to a projection applied by inkjet to the cap D) can be effected by pressing the cap D onto the bellows C. This causes the injection of an amount of gas/fluid from within the bellows into the micro-channels.

For illustrative purposes the device is shown as being rectangular in nature and therefore taking the form of a “slide”. However, different embodiments can be effected with different shapes and sizes to be most appropriate for the application and the needs of a particular testing protocol.

In a second embodiment where the substrate or a component thereof is fibrous in nature and the fibres are intended to provide capillary (wicking) movement of the medium it may be necessary to further control and define the hydrophobic nature of areas around the actual channels; which can be effected by the same computer design method. In this embodiment further capability exists in placing fibrous content into the channels built upon the substrate to provide an additional means of fluidic control of the medium.

An example multistep process for producing a biosensor in accordance with the method of the invention is set out in Table 1.

The process begins at Step A1 where a suitable substrate is received. It should be noted that the substrate is as yet untreated in Step A1. In step A2 a surface treatment is provided to modify the surface wettability of all, or part of the surface. Such modification is most easily effected using corona or plasma discharge systems or chemical treatments such as dipping, spraying or even fludjet application. The master arrangement of micro-channels is then generated at step A3, typical processes for generating the arrangement including stamping, embossing, jetting or otherwise forming the matrix. As a prototyping concept it would be feasible to consider the use of a fluidjet procedure to form the initial master structure and perform the subsequent modification step coincidentally. This idea would have some merit depending on the level of complexity of the system and the output of finished devices required. The master arrangement has the first configuration at step A3.

In step B1 the master arrangement is treated to prepare it to receive the fluidjet material. The treatment at this stage could be a further wettability modification, although at these levels it is more likely that more specific parts of the matrix would be modified using focussed techniques such as lasers or possibly a fluidjet deposition of a primer or coupling agent suited to the substrate chosen. In step B2 the fluidjet process is carried out upon the master arrangement by jetting the fluids to program the structural components of the master arrangement. Once the fluid regions have been deposited they are then cured within step B3.

A further surface treatment of the structure is then performed at step C1. Likewise as noted for step B1 above, this would be imagined as a more specific, targeted modification of sites within the matrix using for example lasers or a fluidjet primer. This prepares the structure for receipt of further jetted material. In step C2, the support components for reagent deposition are then jetted and thereafter cured in step C3. Examples of such support materials would be sol-gel structures or similar inert materials of high specific surface area that may act as a reaction surface.

A further surface treatment as required is then performed in step D1 (similar in nature to those discussed in steps B1 and C1) in preparation for the provision of the reagent material. At step D2 a precisely metered amount of one or more reagents are provided to predefined locations within the master arrangement. Such locations typically include chambers. The reagents are preferably also provided by the utilisation of a fluidjet process. In step D3, the micro-channels are sealed by the application of a sealing layer. The sealing may include the provision of a sealed in amount of air or other gas.

In step E1 the upper sealed surface is treated and thereafter lenses are attached to the locations at which the reagent is to be examined in step E2. An optional curing step is applied at step E3 if the adhesives used to attach the lenses require a cure. At this stage the biosensor is functionally complete.

Step F1 includes separately surface treating a material to form an outer layer for the structure. This is then fluidjet printed in step F2 to provide various batch, usage and other data. The fluidjet material is then cured at step F3. At step G1 the outer layer and the structure are brought together, followed by a trimming and forming step G2. The outer layer and structure are then glued. Any further functional elements are then added in step G3 (such as a multifunctional cap).

In the final steps, at H1 non-functional elements are added. A quality control process is performed at step H2. Finally the completed biosensor is packaged at step H3 and is then ready for use.

TABLE 1

| Step A | | Step B | | Step C | | Step D | |
|---|---|---|---|---|---|---|---|
| 1 | Receive the | 1 | Surface treat the | 1 | Surface treat the | 1 | Surface treat for the |
| | substrate without | | substrate and master | | master matrix for | | master matrix for the |
| | the master matrix | | matrix for jetting of | | programming the flow | | deposition of reagents |
| | | | fluids | | characteristics of the | | |
| | | | | | already partially | | |
| | | | | | programmed matrix | | |
| 2 | Surface treat the | 2 | Jet the fluids to | 2 | Jet required support | 2 | Deposit a metered |
| | substrate | | program the master | | components ready for | | amount of one or |
| | | | matrix structural | | reagent deposition | | more reagents in |
| | | | components | | | | designated locations |
| 3 | Stamp, emboss, jet | 3 | Cure the fluids | 3 | Cure the fluids | 3 | Apply sealing layer |
| | or otherwise create | | | | | | and seal, inserting any |
| | the master matrix | | | | | | gas or air intended in |
| | | | | | | | the design |

| Step E | | Step F | | Step G | | Step H | |
|---|---|---|---|---|---|---|---|
| 1 | Surface treat the | 1 | Surface treat the | 1 | Bring together active | 1 | Add by assembly any |
| | upper surface | | material for the | | element of the sensor | | desired embellishments |
| | | | outer layer | | with outer layer | | of a non functional |
| | | | | | | | nature |
| 2 | Deposit the lenses | 2 | Print by jetting the | 2 | Form and trim, or | 2 | QC process |
| | at defined | | outer enclosure with | | trim and form Glue | | |
| | locations | | necessary batch and | | | | |
| | | | use information, | | | | |
| | | | Pharma code etc | | | | |
| 3 | Cure the fluids if | 3 | Cure the fluids | 3 | Add by assembly any | 3 | Pack |
| | necessary without | | | | functional embellish- | | |
| | affecting the | | | | ments such as a | | |
| | reagent | | | | multi purpose cap | | |

In all embodiments, the principal method of manufacture utilises fluidjet (and in particular inkjet) printing for the manufacture or programming of structures and then subsequent placement of the reagents. According to the nature of the device and the quantity to be produced, the manufacturing process may integrate other methods such as stamping, embossing, laser cutting and other processes in common use within the print and converting industries.

The raw substrate for forming the master arrangement may have been manufactured in bulk with the master arrangement included at a different time to the programming steps and completion of manufacture (such as in Step A1 to A3 above). This is done simply to optimise the economics of the production and can equally be carried out inline with the remainder of the production process. Step A therefore maybe offline or inline to the remainder of the process. In a typical embodiment, the process steps are defined in order from Step A1 to Step H3. Within each step some stages may not be necessary according to the specificity required and the techniques used within each stage with the steps may be different (for example Step A2 may be corona or plasma, while Step D1 may be by laser patterning). Furthermore each of Step A to Step H may require different running speeds when intermediate buffering of partially manufactured product may be required, or the processes are duplicated to achieve a common process speed.

In a simple example it is possible to consider one unit (a sensor in manufacture) following another in a serial manner down the process production line but in a more productive embodiment the present invention allows for production of units in parallel.

In a simple embodiment the Steps G and H may also be offline according to the required production rates and complexity of process. In a more productive embodiment the present invention allows for them to be inline.

The manufacture of these multichannel devices can be considered as a stepwise process where certain production machines are used to carry out one operation, or a series of operations, before passing the device onto the next production machine. Most preferably these machines are arranged as a production line, with partially assembled devices moving on, for example conveyors, between each machine.

Conceptually then the device production can be broken down into several stages as follows:

1) Production and programming the master arrangment.
2) Placement and encapsulation of reagents and other functional materials.
3) Deposition of lenses, other reporting interfaces.
4) Sealing and packaging of device.
5) Quality Control testing and final pack.

These steps are more fully described below. It should be understood that any of the above stages may include a batchwise production step (for example certain lots may require steam sterilisation at some stage). Likewise, steps 4 & 5 could conceivably be offline, manual processes depending on required production rate or process complexity.

In each of the above processes it should be considered that a multiplicity of parts is produced and the example below is only considered as an illustration for a single part.

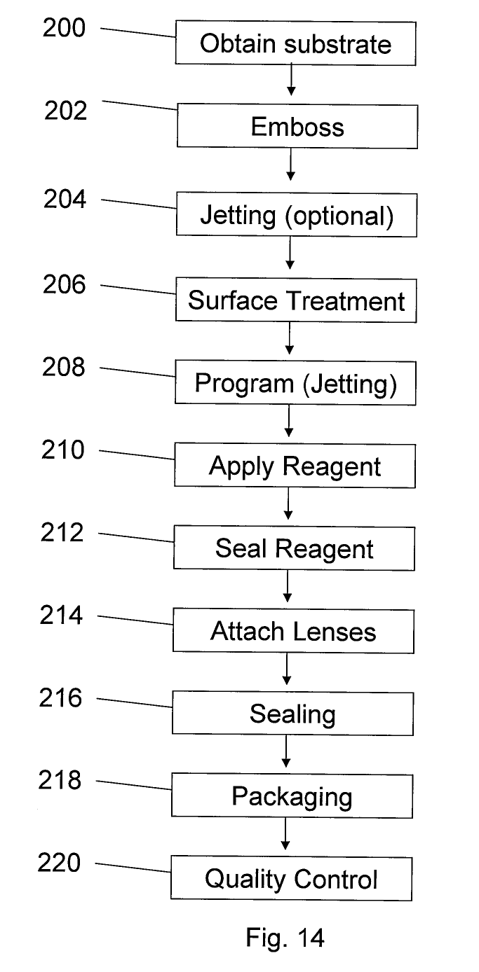

Using a basic ELISA (Enzyme-Linked ImmunoSorbent Assay) as an example, a stepwise production process can be more easily explained as follows with reference to the flow diagram of FIG. 14. This provides further explanation of the generalised process set out in Table 1.

As a first step 200, a fibre based stock is chosen for its flow/absorbent properties for this specific application as the substrate. More particularly this material could be selected from a filter paper grade, or manufactured from paper and wood pulp or selected cellulose/hemi cellulose mixtures to give pre-defined areas of wetting.

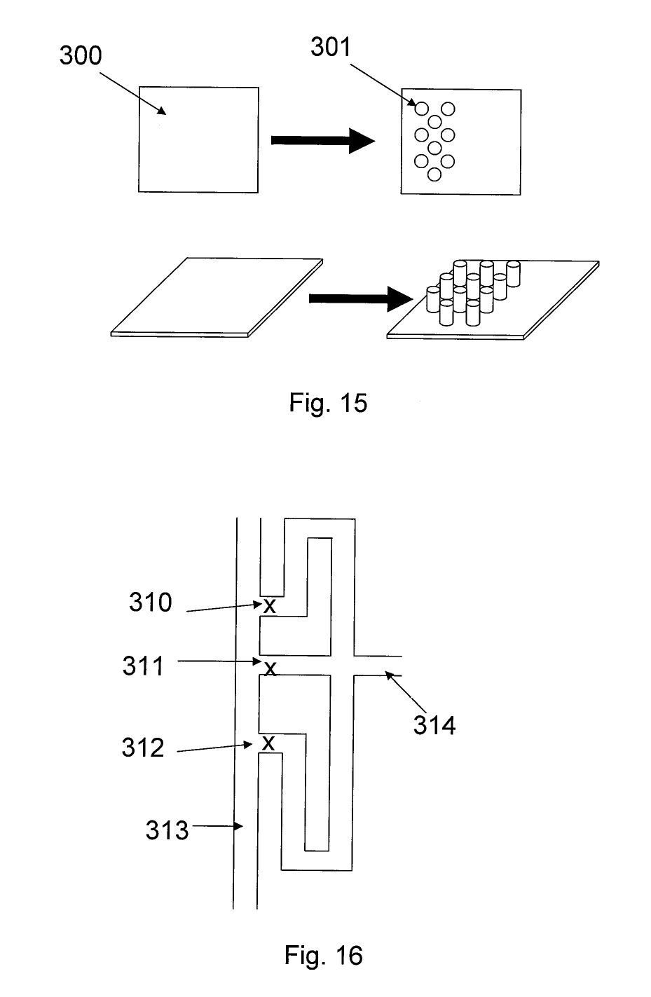

At step 202 the substrate stock material is pressed/embossed using a standard procedure to define the flow channels, reservoirs, mixing chambers and detection zones (collectively referred to as micro-channels herein) required for a common ELISA process. It should be considered that in a more complex application this embossing step could define several copies of a similar configuration so that a number of different ELISA tests could be included on the same device. If necessary, modifications or additions to the embossed pattern could be made using a jetting process at step 204. For example a simple embossed zone could be converted into a mixing chamber by the addition of jetted pillars at carefully arranged spacing. This is illustrated in FIG. 15 (in plan and perspective schematic views) where a master arrangement 300 (an embossed substrate) is provided with a number of closely spaced cylinders of jetted material.

Depending on the medium being tested, it is likely that the flow channels and all defined areas will be treated to alter the wetting characteristics. This could either be effected by masking and a coating process, or inkjet application of the modifying fluid, or by using corona discharge/laser activation or similar surface modification technique. Such a step is performed at 206.

At step 208 a programming step occurs so as to modify the configuration of the master arrangement of micro-channels. This is performed using fluidjet methods and in the specific ELISA example is used to alter the flow paths and hence residence time of certain materials. For example an antibody/antigen interaction may require a specific reaction period before a flushing step, and therefore the flow path of a buffered flush could be altered by jetting a phase change material into specific flow paths. This is illustrated in FIG. 16 where three alternative channels 310, 311, 312 each link a first channel 313 to a second channel 314. In this example, the flush could travel through three different flow paths representing different delay times. Placement of an obstruction (such as by jetting a phase change material) into two of the positions marked by a “X” will program in a specific delay.

In a similar manner to the configuration of the master arrangement, the preferred placement process for reagents, flushes, and other functional materials (for example, specifically designed sensor/substrate molecules) is by utilising a fluidjet technique due to its inherent placement accuracy and drop volume control. This technique can therefore be used to place precisely metered amounts of buffered flush, primary antibody material, conjugate material (used in certain ELISA tests if an enzyme-linked version of the primary antibody does not exist or is difficult to produce), and substrate (sensor molecule, usually a chromogenic compound). The various fluids are typically delivered in an appropriate solution to aid jettability, but minimise unwanted liquid movement (by absorption or spreading). The fluids thus manufactured would benefit from proven stability commensurate with the period of use in the production system. Such a jetting step to provide the reagent occurs at step 210.

Depending on the assays being constructed a number of different multiplex jetting systems could be used utilised:

A) Multiple single nozzles (similar to the MicroFab™ type) jetting different fluids at the same time;
B) Specific print head types capable of jetting different fluids—such as a Xaar XJ500 print head;
C) A single print head with multiple nozzles applying the same fluid into specific areas (an example of this for the ELISA example might be the placement of buffered flush into specific reservoir areas).

An encapsulation step 212 may be necessary to prevent early activation of a particular reagent, or to prevent evaporation, or as a protection for a subsequent step. Again, the application of specific amounts of an encapsulant in defined locations lends itself to a fluidjet process. The encapsulant thus applied must be chosen to dissolve or otherwise react with the test medium containing the analyte of interest, so that the underlying reagent or sensor molecule can interact with the analyte without adverse competition.

To improve the signal/noise ratio or to improve the readability of the chromogenic (for example, for ELISA) or fluorogenic molecules, a plastic lens can then be deposited over the detector wells originally embossed in the fibre media. This technique has previously been demonstrated for capping fibre optic cables by using a MicroFab™ system and it is therefore suggested a similar technique be adopted herein. Such lenses may are applied at step 214. Other reporting devices, such as conductive tracks for powered pumps, switching valves, or RFID antenna could also be added at this stage. Additionally this stage could also include the application (by fluidjet) of tracking information, such as a bar code.

A sealing step 216 is then applied. This could be considered a simple lamination process utilising a similar fibre product as was used to form the substrate. This could also have certain flow areas embossed into it, or areas of wettability pre-defined. Additionally the detection windows are cleared on the top surface prior to lamination. The actual process of lamination preferably involves gluing or pressure sealing, as a thermal process may affect the stability of the pre-applied components. Alternatively a plastic sealing layer could be applied by any traditional method including curtain coating, spraying, and roller coating.

Once the device has been sealed a further packaging step 218 may be considered to provide a degree of instrumental presentation and user interactivity. Specifically for ELISA analysis a ‘dip’ probe or similar sampling point would be need to be made available for urine tests. Also, depending on the nature of the fluid to be tested, a primary (coarse) filter matrix may be included to remove potential contaminants.

Due to the nature of the product, a statistically relevant proportion of the manufactured volume will have to be quality control tested. This would potentially be an offline process depending on the complexity of the tests involved. This also would be the case in the final packaging of the product, as this would critically depend on the form factor and function of the test device. This quality control testing occurs at step 220 in FIG. 14.

The methods described therefore provide a flexible and low cost means for effecting bio-sensing devices having tailored specificity.

1-26. (canceled)

27. A method of forming a micro-channel structure for use in a biosensor, comprising:
a) providing a master structure having a first configuration of micro-channels with respective first fluid flow characteristics; and,
b) depositing one or more regions of material onto the master structure using a fluidjet process so as to modify the first configuration into a second configuration having respective second fluid flow characteristics, different from the first.

28. A method according to claim 27, wherein one or more of the micro-channels comprise one or more of conduits, valves and chambers.

29. A method according to claim 27, wherein the modification of the first configuration comprises blocking one or more of the micro-channels.

30. A method according to claim 27, wherein the modification of the first configuration comprises partially restricting one or more of the micro-channels.

31. A method according to claim 27, wherein the micro-channels are provided as a plurality of sets of one or more micro-channels and wherein the arrangement of the micro-channels is the same within each set.

32. A method according to claim 31, wherein the sets of micro-channels are arranged side-by-side in an array.

33. A method according to claim 32, wherein the sets of micro-channels are arranged in a two-dimensional or three-dimensional array.

34. A method according to claim 27, wherein, following the deposition of the fluidjet material to form the second configuration, the method further comprises applying a curing treatment to the deposited material.

35. A method according to claim 27, further comprising applying one or more further processes so as to provide a biosensing function.

36. A method according to claim 35, wherein each set of micro-channels is further processed to provide a similar biosensing function.

37. A method according to claim 35, wherein two or more sets of micro-channels are further processed to provide different biosensing functions.

38. A method according to claim 35, wherein the providing of the biosensing function further comprises providing a reagent to at least one of the micro-channels for use in a biosensing function.

39. A method according to claim 38, wherein the reagent is provided by a fluidjet process.

40. A method according to claim 39, wherein the reagent and the material of step (b) is provided using the same fluidjet apparatus.

41. A method according to claim 27, further comprising applying a surface treatment to one or more regions of the micro-channels so as to affect the wettability characteristics of the said one or more regions.

42. A method according to claim 27, further comprising, applying a sealing layer to the substrate to as to enclose the said micro-channels.

43. A method according to claim 27, further comprising applying an optical element to the structure, the optical element comprising one or more or a lens, waveguide, light pipe or grating.

44. A configured micro-channel structure for use in a biosensor, comprising a master structure having a first configuration of micro-channels and one or more regions of fluidjet deposited material applied to the micro-channels so as to provide a second configuration in which the fluid flow characteristics of the master structure are modified.

45. A biosensor comprising a micro-channel structure according to claim 44, wherein the micro-channel structure is provided with an immobilised reagent, and a delivery system for providing an analyte to the reagent.

46. A biosensor according to claim 45, further comprising a bellows adapted to provide an amount of a predetermined gas or liquid to the micro-channel structure.

47. A biosensor according to claim 45, further comprising a removable cap for exposing a region for provision of an analyte-bearing medium to the biosensor or for supporting the biosensor when in a predetermined orientation when in use.

48. A biosensing device according to claim 46, wherein the biosensing device further comprises a cap having a projection and wherein the cap is adapted to enable the bellows to be deformed in a predetermined manner.

49. A biosensing device according to claim 45, adapted to only utilise capillary flow or fibre wicking in order to initiate the analyte coming into the necessary proximity with the reagent.

50. A micro-channel structure manufactured using a method according to claim 27.

51. A micro-channel structure according to claim 44, wherein the master structure is formed within a substrate comprising a material selected from the group of: a naturally fibrous paper or board; constructed fibrous material that contains a designed and oriented arrangement of one or more of cellulose, hemi cellulose and lignin; a material formed by a polymerisation process; or granulated glass.

52. A computer program comprising program code means adapted to cause a computer to perform the method of claim 27, when executed upon a computer.
